(19) United States
(12) Patent Application Publication
(10) Pub. No.: US 2022/0386877 A1
(43) Pub. Date: Dec. 8, 2022

(54) PROXIMITY-TEMPERATURE SENSOR PACKAGE AND EARPHONE APPLICATION

(71) Applicant: Luxsentek Microelectronics Corp., New Taipei City (TW)

(72) Inventors: WEN-SHENG LIN, New Taipei City (TW); SHENG-CHENG LEE, New Taipei City (TW); CHIH-WEI LIN, New Taipei City (TW); CHEN-HUA HSI, New Taipei City (TW); YUEH-HUNG HOU, New Taipei City (TW)

(21) Appl. No.: 17/479,210

(22) Filed: Sep. 20, 2021

(30) Foreign Application Priority Data
Jun. 3, 2021 (TW) 110206409

(51) Int. Cl. A61B 5/01; G01S 17/08; H04R 1/10; H04R 1/04

(52) U.S. Cl. CPC A61B 5/01; G01S 17/08; H04R 1/1016; H04R 1/04

(57) ABSTRACT

The present invention proposes a packaging structure that integrates distance sensing device and temperature sensing device, and it is applied to small wearable devices such as earphones or watches. The combination of distance and temperature sensing allows the wearable device to accurately determine whether it is on the user's body, and then start temperature monitoring. It can collect ear temperature information more effectively and energy-saving.

### BACKGROUND OF THE INVENTION

### 1. Field of the Invention

The present invention relates to a sensor package and particularly relates to the proximity-temperature sensor package and its earphone application.

### 2. Description of the Prior Art

A Bluetooth earphone usually integrates a proximity sensor (PS) to detect wearing status to turn on/off the Bluetooth chip for reducing power loss. Moreover, it is developing to use earphones to collect physiological information such as ear temperature, heart rate, and blood oxygen. More and more sensors are put together, and their interference for each other is an important issue.

Thin, small, wireless, low consumption, anti-interference and good look are required to a wearable electronic product. That means it is necessary to be light, miniaturized, and invisible for the sensors.

The invention proposes an integrated package of a temperature sensor and a proximity sensor. The integrated package avoids/reduces crosstalk and environmental interference, and meets the requirements of lightness, thinness, and invisibility.

### SUMMARY OF THE INVENTION

The present invention provides a packaging structure to integrate a proximity sensor (PS) and a temperature sensor (TS), and its earphone application. The proximity sensor is configured to measure the distance to an object and the TS is configured to measure the ear temperature. The packaging structure uses a light blocking member to separate the light-emitting element and the photosensitive element of a PS to avoid or reduce crosstalk. An opaque layer is set around the TS to avoid or reduce interference from the light.

The present invention provides packaging structure comprising:

a light-emitting unit disposed on a first end of a surface of a substrate:

a photosensitive element and a temperature sensing element disposed on a second end of the surface of the substrate;

an opaque layer covered the temperature sensing element; and

a transparent package housing covered the substrate, the light-emitting unit, the photosensitive element, the temperature sensing element and the opaque layer, wherein the transparent package housing has a groove between the first end and the second end, the groove extends toward the substrate and is filled with opaque material.

The earphones can use the TS to monitor user's body temperature and remind user the physiological conditions or report the medical measurements.

### BRIEF DESCRIPTION OF THE DRAWINGS

FIG. 1 is a side view of the packaging structure of an embodiment of the present invention.

FIG. 2 is a top view of the packaging structure of an embodiment shown as FIG. 1.

FIG. 3 is a side view of the packaging structure of another embodiment of the present invention.

FIG. 4 is a side cross-sectional view of the earphone of the present invention.

### DESCRIPTION OF THE PREFERRED EMBODIMENTS

Below embodiments accompanied with drawings are used to explain the spirit of this invention to have better understanding for the person in this art, not used to limit the scope of this invention, which is defined by the claims of this patent application. The applicant emphasizes the element quantity and size are schematic only. Moreover, sonic parts might be omitted to skeletally represent this invention for conciseness.

The invention provides a packaging structure with a proximity sensor and a temperature sensor. The height H of the packaging structure is between 0.5 mm and 0.7 mm and that satisfies the requirements of wearable devices such as earphones or watches.

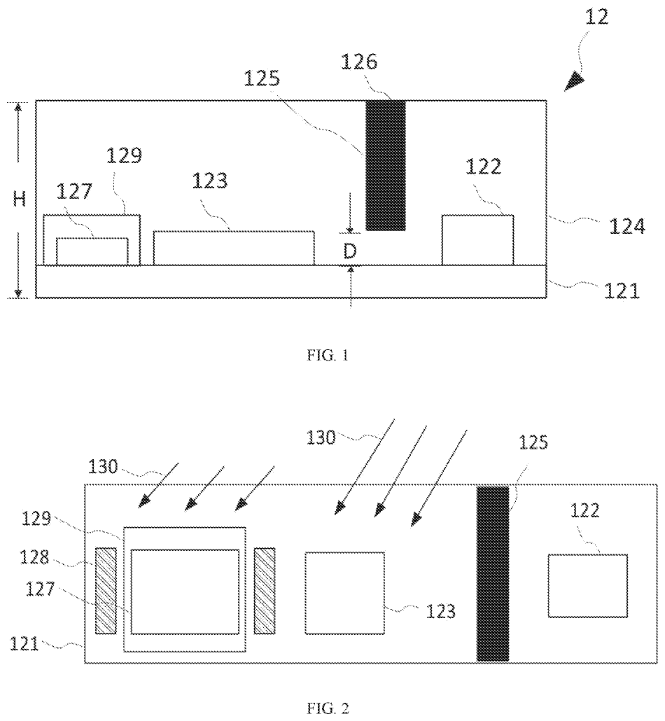

A proximity sensor and a temperature sensor directly disposed on a substrate 121. FIG. 1 and FIG. 2 respectively schematically show a side view and a top view of the packaging structure 12.

The packaging structure 12 comprises a light-emitting unit 122 is disposed on a first end of a surface of the substrate 121, and a photosensitive element 123 and a temperature sensing element 127 are disposed on a second end of the surface of the substrate. An opaque layer 129 covers the temperature sensing element 127. A transparent package housing 124 covers the substrate 121, the light-emitting unit 122. the photosensitive element 123, the temperature sensing element 127 and the opaque layer 129, wherein the opaque layer 129 covers on the temperature sensing element 127. The transparent package housing 124 has a groove 125 between the first end (the light-emitting unit 122) and the second end (the photosensitive element 123 and the temperature sensing element 127), and the groove 125 extends forward to the substrate 121 and filled with opaque material 126. The opaque material 126 can avoid the light interference of the light-emitting unit 122 to the photosensitive element 123 and the temperature sensing element 127.

FIG. 2 is a top view of an embodiment. The opaque layer 129 covers on the temperature sensing element 127 to prevent or reduce the interference from the light 130. In addition, isolation layers 128 are disposed on both sides or around of the temperature sensing element 127. The isolation layers 128 can be N-wells or deep N-wells to prevent from or reduce the circuit noise. The thickness of the opaque layer 129 can he 0.6 μm˜1.0 μm, and the width can be 8 μm˜12 μm. The depth of the N-wells as the isolation layers 128 can be 0.6 μm˜1.0 μm, and the depth of deep N-wells can be 2.8 μm˜3.2 μm.

The width of the groove 125 is 80 μm˜120 μm and a distance D between the groove 125 and the surface of the substrate 121 is 20 μm˜100 μm. The distance D should be small enough to avoid or reduce light crosstalk from the light-emitting unit 122 to ensure the function of the photosensitive element. The material of the transparent package housing 124 is a low molecular weight epoxy resin, and its optical. refractive index is between 1.55 and 1.65.

The light-emitting unit 122 can be implemented by a vertical-cavity surface-emitting laser (VCSEL) or a light-emitting diode (LED). The light-emitting unit 122 is configured to have a smaller light divergence angle, so it effectively reduces light scattering and propagation to reduce the crosstalk interference. The opaque layer 129 or the opaque material 126 is silicon wafer, metal, epoxy resin, resin-silicone glue or acrylic glue or combination thereof.

In an embodiment, the surface of the substrate 121 is optionally covered with a black layer or a material, this is designed to have a high light absorptivity to avoid/reduce the reflection.

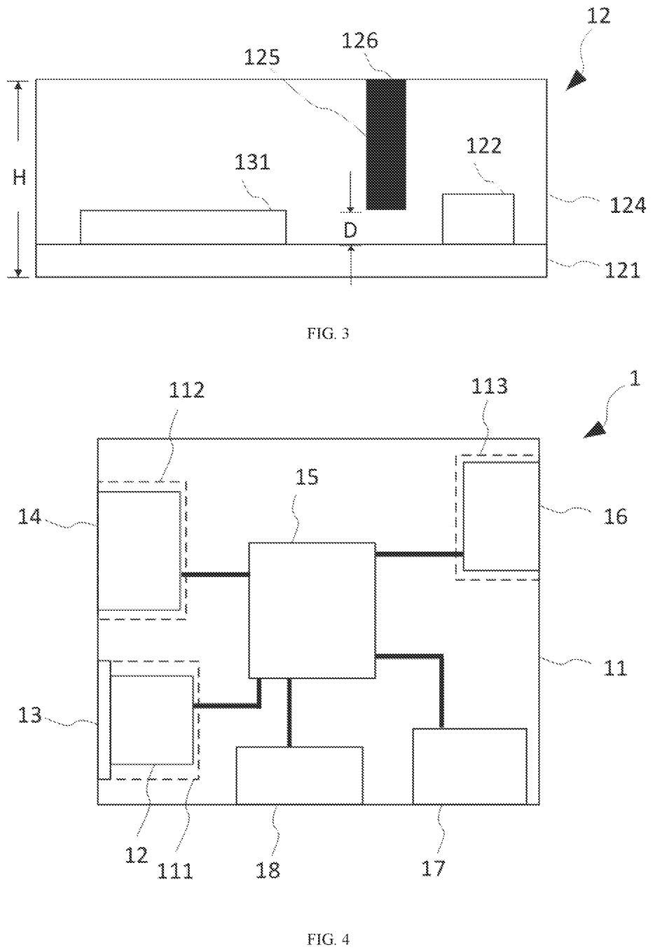

In an embodiment, the photosensitive element 123 and the temperature sensing element 127 can be integrated into a single composite element to reduce the package volume, as shown in FIG. 3. The temperature sensing element 127 with the opaque layer 129 is stacked on the photosensitive element 123 (not shown in the figure). The temperature sensing element 127 with the opaque layer 129 is still smaller than the photosensitive element 123, and it does not affect the photosensitive function.

FIG. 4, a side cross-sectional view of an accommodation space of a housing 11 of an earphone 1, shows the layout of the packaging structure 12 and other components, such as an audio playing unit 14, a control unit 15, and a microphone unit 16. The packaging structure 12 is disposed in a sensing hole 111 and covered by a sensing cover 13. The sensing cover 13 is coplanar with the surface of the earphone 1. The packaging structure 12, the audio playing unit 14, and the microphone unit 16 are electrically connected to the control unit 15.

In general, the material of the sensing cover 13 is selected to be low or opaque to visible light but partially penetrated by the laser emitted by the light-emitting unit 122. Therefore, the detection light from the light-emitting unit 122 and its reflection light can go in and out to reach the photosensitive element 123. In addition, the sensing cover 13 is non-porous and its color can be selected to be consistent with the earphone surface to maintain the integrity design.

For the audio module, the audio playing unit 14 is placed in an audio hole 112, and a microphone unit 16 in the microphone hole 113. The audio playing unit 14 and the microphone unit 16 are controlled by the control unit 15.

The earphone 1 further comprises a communication unit 18 connected to the control unit 15, configured to communicate with an external electronic device by wire or wirelessly. In one embodiment, the communication unit 18 is controlled by the control unit to transmit the sensed distance and temperature information to the external electronic device. The earphone 1 further comprises a battery unit 17, which is configured to supply power to proximity-temperature sensor, the control unit 15 and other components.

The invention proposes a proximity and temperature sensor integration package. It means the package has more function but smaller volume. Especially, the non-porous sensing cover can enhance anti-dust and design flexibility.

What is claimed is:

1. A packaging structure with proximity and temperature sensing functions, comprising:
a light-emitting unit disposed on a first end of a surface of a substrate;
a photosensitive element and a temperature sensing element disposed on a second end of the surface of the substrate;
an opaque layer covered the temperature sensing element; and
a transparent package housing covered the substrate, the light-emitting unit, the photosensitive element, the temperature sensing element and the opaque layer, wherein the transparent package housing has a groove between the first end and the second end, the groove extends toward the substrate and is filled with opaque material.

2. The packaging structure according to claim 1, wherein a width of the groove is 80 μm to 120 μm, and a distance of the groove to the surface of the substrate is 20 μm to 100 μm.

3. The packaging structure according to claim 1, wherein the light-emitting unit is a vertical-cavity surface-emitting laser or a light-emitting diode.

4. The packaging structure according to claim 1, wherein the surface of the substrate is covered with a black layer.

5. The packaging structure according to claim 1, wherein N-wells or deep N-wells are disposed on both sides or around of the temperature sensing element.

6. The packaging structure according to claim 1, wherein the opaque layer or the opaque material is silicon wafer, metal, epoxy resin, resin-silicone glue or acrylic glue or combination thereof.

7. The packaging structure according to claim 1, wherein the photosensitive element and the temperature sensing element are integrated into a single composite element.

8. The packaging structure according to claim 1, wherein a height of the packaging structure is between 0.5 mm and 0.7 mm.

9. An earphone with the packaging structure according to claim 1, comprising:
a housing with an accommodation space;
the packaging structure, an audio module and a control unit disposed in the accommodation space; and
a sensing cover covered the accommodation space to be coplanar with the housing surface, wherein the packaging structure and the audio module are electrically connected the control unit.

10. The earphone according to claim 9, wherein the sensing cover is non-porous.
